(19) United States
(12) Patent Application Publication
(10) Pub. No.: US 2017/0290539 A1
(43) Pub. Date: Oct. 12, 2017

(54) POLYGRAPH

(71) Applicant: REFLECTION TECHNOLOGIES LTD., Tel Aviv (IL)

(72) Inventors: Eyal YAFFE-ERMOZA, Jerusalem (IL); Ovad GOLAN, Jerusalem (IL); Zeev DANIELI, Jerusalem (IL); Ohad PERETS, Jerusalem (IL); Shaul BAR LEV, Tel Aviv (IL); Roni SIVAN, Tel Aviv (IL)

(21) Appl. No.: 15/508,115

(22) PCT Filed: Sep. 2, 2015
(86) PCT No.: PCT/IL2015/050881

§ 371 Date: Mar. 2, 2017

Related U.S. Application Data
(63) Continuation of application No. 14/476,828, filed on Sep. 4, 2014.

(51) Int. Cl. A61B 5/16; A61B 5/00; A61B 5/0205

(52) U.S. Cl. CPC A61B 5/164; A61B 5/0205; A61B 5/6826; A61B 5/0533

(57) ABSTRACT

A method comprising measuring skin conductivity level of a human subject responsive to a series of preliminary questions; measuring skin conductivity level of the human subject in response to a series of control questions; and in case that a sympathetic activation is detected, finding the authenticity of the response to the control question by comparing the recovery time of skin conductivity level for the control question with the basic recovery time. Further, a method comprising measuring heartbeat of a human subject responsive to a series of preliminary questions to which the answer is known, to derive a level of coherence of the heartbeat being a baseline heartbeat coherence level; presenting the human subject with a series of control questions, and finding the authenticity of a response to a relevant question by comparing the heartbeat coherence detected with the baseline heartbeat coherence and the unbalanced heartbeat coherence.

### CROSS-REFERENCE TO RELATED APPLICATIONS

This application claims priority from U.S. application Ser. No. 14/476,828, filed on Sep. 4, 2014.

### FIELD OF THE INVENTION

The present invention relates generally to veracity testing, and more specifically using multisensory data for increasing reliability such testing.

### BACKGROUND OF THE INVENTION

The following publications are considered to represent the current state of the art: U.S. Pat. Nos. 7,972,140; 7,967,750; 7,831,061; 7,822,783; 7,431,700; 6,873,716; 6,339,715; 6,062,216; 5,892,575; 5,792,049; 5,507,291; 5,467,122; 5,278,403; 5,241,360; 5,142,372; 4,289,142; 4,123,160; 4,085,740; 3,230,951; 2,944,542; Re: 33,865; U.S. Patent Application Publication No. 2004/0031906; and PCT Patent Application Publications Nos. WO/2010/104480; WO/2010/092366; and WO/2008/029121.

### SUMMARY OF THE INVENTION

Some embodiments of the present invention may provide a method for polygraph analysis, the method may include: measuring skin conductivity level of a human subject responsive to a preliminary questioning comprising a series of preliminary questions, wherein the measured skin conductivity level is defined as a baseline skin conductivity level.

Further, the method according to embodiments of the present invention may include presenting the human subject with a sensual stimulation which is sufficient to derive an autonomous nervous system of the human subject into a sympathetic activation and measuring the skin conductivity level of the human subject after said sympathetic activation.

The measured skin conductivity level in response to the preliminary questions is defined according to embodiments of the present invention as stress basic response level, and a basic recovery time is defined as a time after which the value of the measurement reaches a specified value between the stress basic response level and the baseline skin conductivity level.

Further, the method according to some embodiments of the present invention may include measuring skin conductivity level of said human subject in response to a series of control questions to which the answer is known to the subject and non-controversial, and in case that a sympathetic activation is detected, finding the authenticity of the response to the control question by comparing the recovery time of skin conductivity level for the control question with the basic recovery time.

In some embodiments of the present invention, the measuring of the skin conductivity level of the human subject is carried out using a skin conductivity measurement device having a variable resistance that is matched to the resistance of said human subject.

In some embodiments of the present invention, the preliminary questioning includes at least two questions with a known non-controversial answer.

In some embodiments of the present invention, the method further includes after presenting the stimulation, deduction of the automatic response level and the regular behavior of the subject under the presented questioning conditions.

In some embodiments of the present invention, the method further includes, during the responding to a series of control questions, comparing the measured values to the baseline response levels and the recovery time after each response to the basic recovery time.

In some embodiments of the present invention, the method further includes measuring heartbeat coherence of the human subject during at least the series of control questions and calculating the subject's heartbeat coherence baseline structure.

In some embodiments of the present invention, the method further includes measuring the fluctuation degree of the skin conductivity, and if the fluctuation degree is higher than a certain predetermined threshold of fluctuation and the heartbeat coherence is higher and/or substantially different in structure than the heartbeat coherence baseline, indicating the responses to the control questions as suspected as non-authentic.

Further, some embodiments of the present invention may include measuring heartbeat of a human subject responsive to a series of preliminary questions to which the answer is non-controversial and known to the subject, to derive a level of coherence of the heartbeat being a baseline heartbeat coherence level.

In some embodiments of the present invention, the preliminary questioning includes at least two questions with a non-controversial known answer.

In some embodiments of the present invention, during the preliminary questions stage, the method further includes learning the basic heart coherency of the specific subject, and during the stage of control questions, the method further includes learning the stress response coherency.

Further, the method according to some embodiments of the present invention may include presenting the human subject with a series of control questions to which the answer has variable length, wherein the control questions are being asked in a rate that does not allow said human subject to selectively control the subject's respiratory system, so that the heartbeat coherence level of said human subject becomes unbalanced.

Further, the method according to some embodiments of the present invention may include measuring heartbeat of the human subject in response to a series of questions relevant to the questioning subject and finding the authenticity of a response to a relevant question by comparing the heartbeat coherence detected with the baseline heartbeat coherence and the unbalanced heartbeat coherence. For example, in case that the coherence level in response to a relevant question is better than said baseline heartbeat coherence level, a suspicion may be indicated that the subject's response is non-authentic, e.g. the subject is trying to control his/her heartbeat in order to hide the authentic response to the relevant question.

### BRIEF DESCRIPTION OF THE DRAWINGS

The present invention will be understood and appreciated more fully with reference to the drawings in which:

FIG. 1 is a simplified pictorial illustration of a distributed computerized veracity testing system constructed and operative in accordance with a preferred embodiment of the present invention;

FIGS. 2A and 2B are simplified respective exploded view and assembled view pictorial illustrations of a subject observation subsystem forming part of a computerized veracity testing system constructed and operative in accordance with a preferred embodiment of the present invention;

FIG. 2C is a simplified top view illustration of a subject observation subsystem of FIGS. 2A and 2B;

FIG. 2D is a simplified illustration of hand engagement with the subject observation subsystem of FIGS. 2A-2C;

FIGS. 3A and 3B are simplified sectional illustrations, taken along respective lines IIIA-IIIA and IIIB-IIIB of corresponding FIGS. 2A and 2B;

FIGS. 4A and 4B are simplified sectional illustrations taken along lines IV-IV in FIG. 2D in respective non-immobilized and immobilized operative orientations;

FIG. 5 is a simplified illustration of an alternative embodiment of a subject observation subsystem, forming part of a computerized veracity testing system constructed and operative in accordance with a preferred embodiment of the present invention;

FIGS. 6A, 6B and 6C are simplified illustrations of three different output functionalities employing multiple different types of sensors, preferably provided by the veracity testing system of a preferred embodiment of the present invention;

FIG. 7 is a simplified illustration of the operation of a veracity testing system providing the functionalities illustrated in FIGS. 6A, 6B and 6C;

FIG. 8 is a schematic flowchart illustrating a method for recognizing a non-authentic response of a subject according to some embodiments of the present invention;

FIG. 9 is a schematic flowchart illustrating a method for recognizing a non-authentic response of a subject according to some embodiments of the present invention; and

FIG. 10 is a graph illustration of a method for recognizing a non-authentic response of a subject according to some embodiments of the present invention.

### DETAILED DESCRIPTION OF THE INVENTION

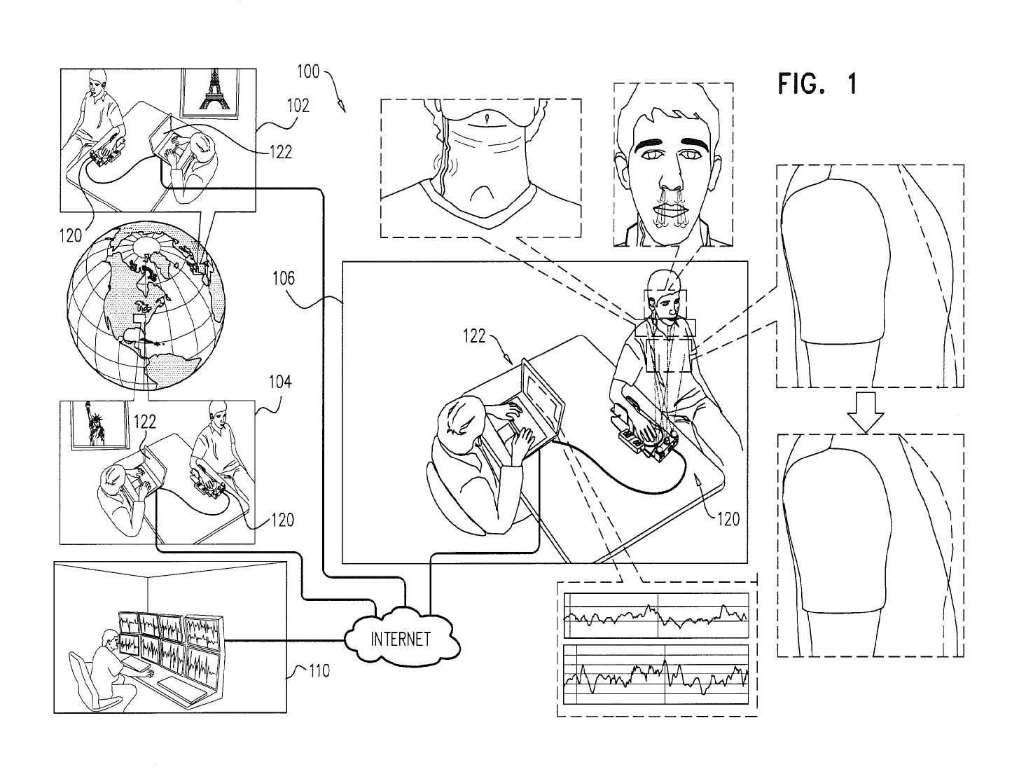

Reference is now made to FIG. 1, which is a simplified pictorial illustration of a distributed computerized veracity testing system constructed and operative in accordance with a preferred embodiment of the present invention.

As seen in FIG. 1, there is provided a computerized veracity testing system generally designated by reference numeral 100. In the illustrated embodiment, the computerized veracity testing system 100 is a distributed system and includes a plurality of veracity testing sites, here designated by reference numerals 102, 104 and 106, which may be interconnected with a veracity analysis center 110, which may be remote from all or some of the veracity testing sites. The various veracity testing sites may communicate unidirectionally or bidirectionally via any suitable data network, such as the internet.

It is appreciated that features described herein below may also be applicable to a non-distributed veracity testing system wherein a single veracity testing site is co-located with veracity analysis functionality.

In accordance with a preferred embodiment of the present invention, at each veracity testing site there is provided a subject observation subsystem including a subject observation assembly, generally designated by reference numeral 120, operative to provide an output representing at least one characteristic of a subject and a data receiving computer 122.

Further in accordance with a preferred embodiment of the present invention, there is provided veracity analysis functionality, which may be, as mentioned above, at a discrete veracity analysis site, such as a veracity analysis center 110, or co-located with a subject observation subsystem, and preferably embodied in a data receiving computer 122.

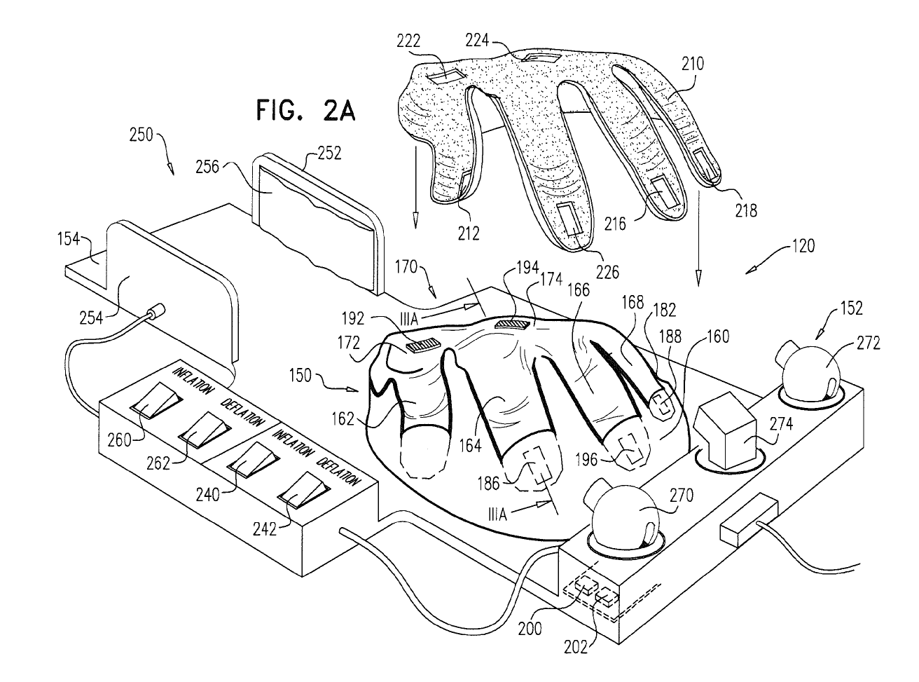

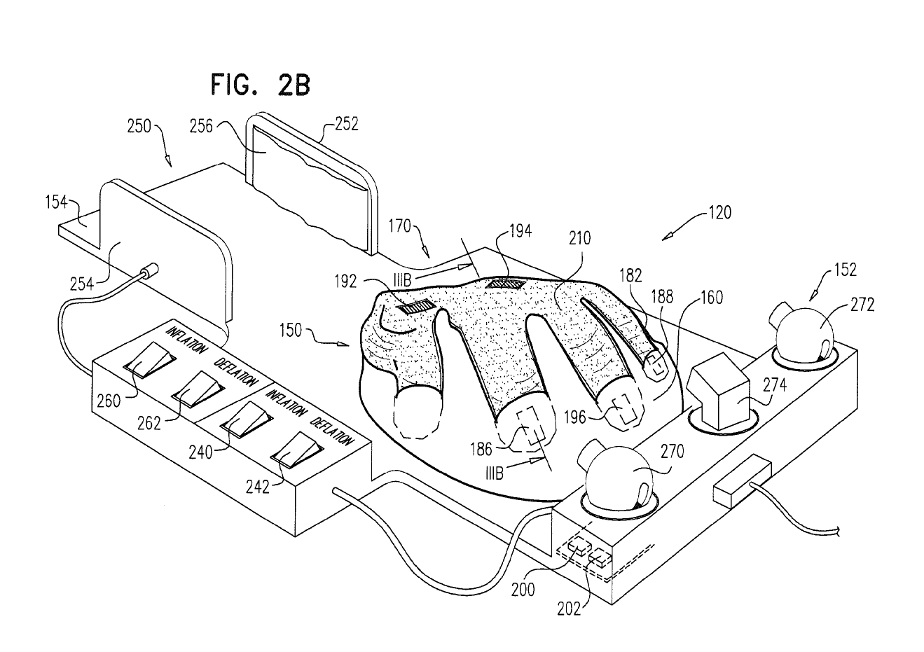

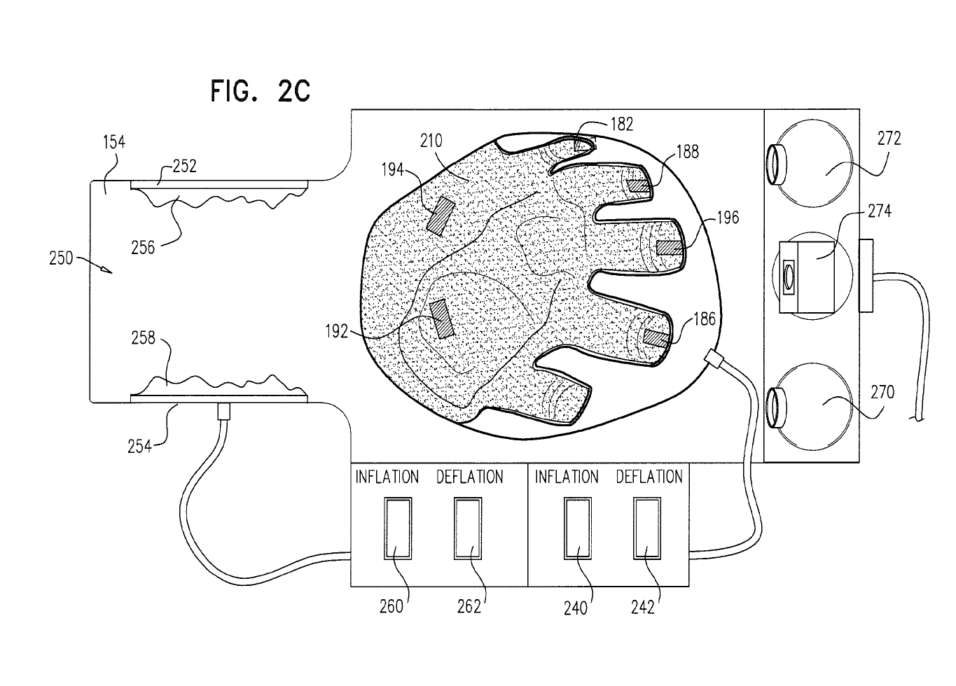

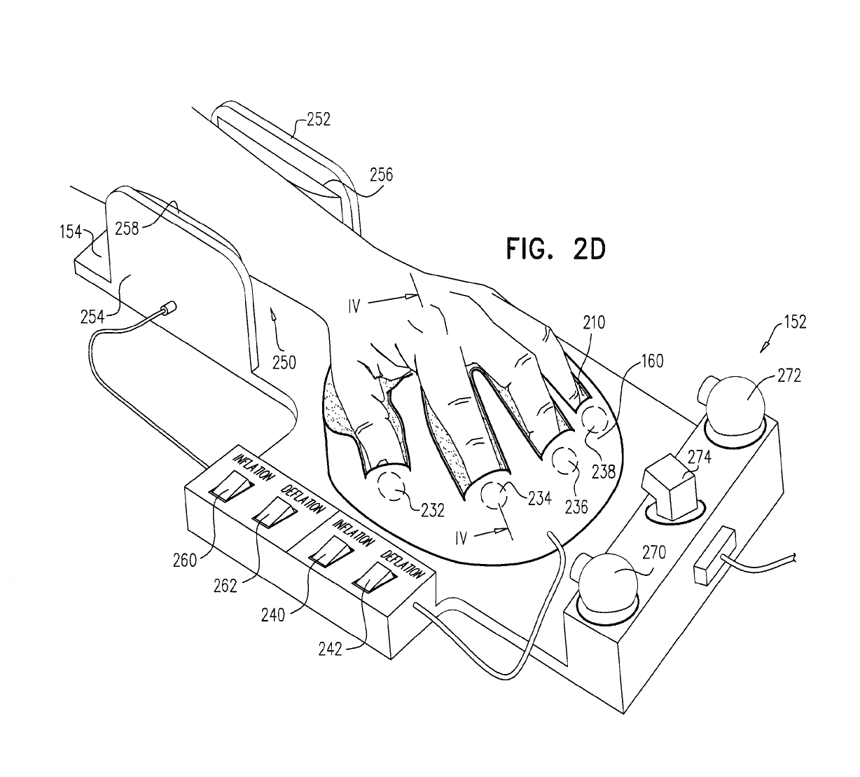

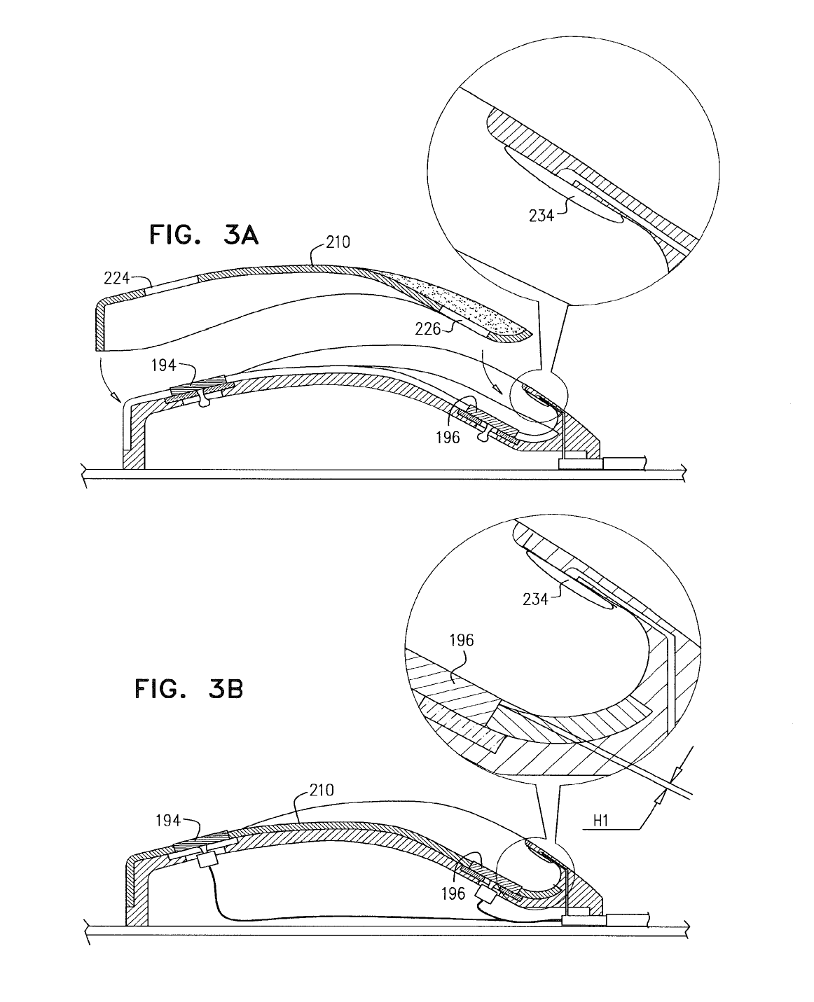

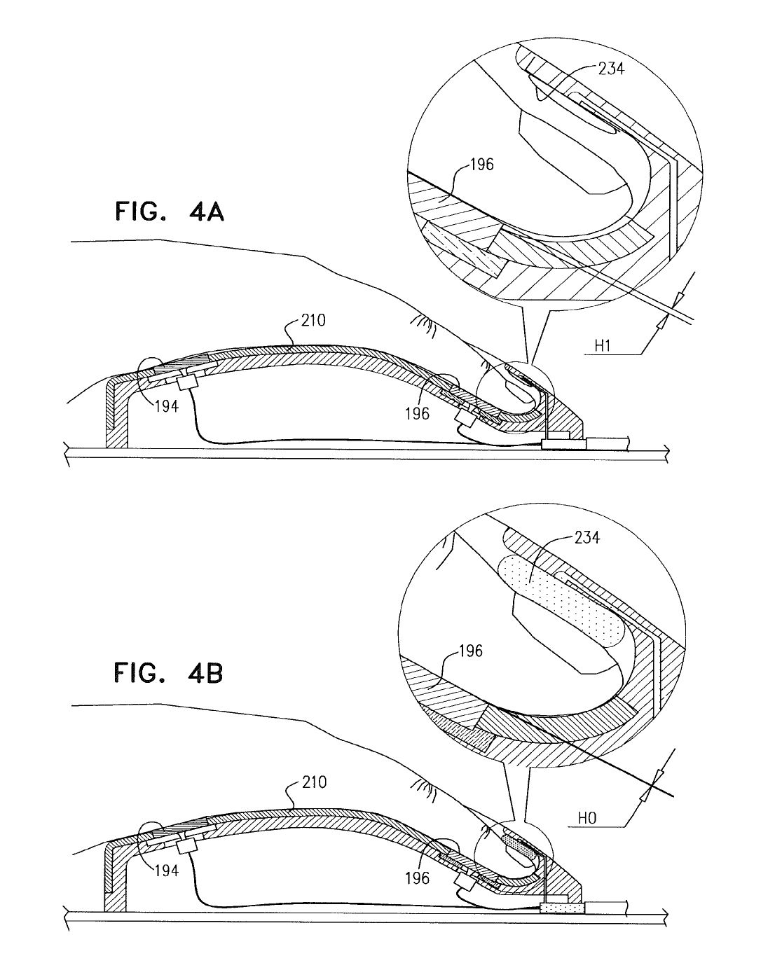

Reference is now made additionally to FIGS. 2A and 2B, which are simplified respective exploded view and assembled view pictorial illustrations of a subject observation subsystem forming part of a computerized veracity testing system constructed and operative in accordance with a preferred embodiment of the present invention, to FIG. 2C, which is a simplified top view illustration of a subject observation subsystem of FIGS. 2A and 2B, to FIG. 2D, which is a simplified illustration of hand engagement with the subject observation subsystem of FIGS. 2A-2C, to FIGS. 3A and 3B, which are simplified sectional illustrations, taken along respective lines IIIA-IIIA and IIIB-IIIB of corresponding FIGS. 2A and 2B, and to FIGS. 4A and 4B, which are simplified sectional illustrations taken along lines IV-IV in FIG. 2D, in respective non-immobilized and immobilized operative orientations.

As seen in FIGS. 1-4B, the subject observation subsystem 120 includes a hand engagement unit 150 and a camera assembly 152, both of which are preferably mounted on a common base 154.

The hand engagement unit 150 preferably comprises an ergonomically shaped hand rest base element 160, which is fixedly mounted onto common base 154. Hand rest base element 160 preferably includes four finger support areas 162, 164, 166 and 168 and a palm support area 170 including first and second palm support regions 172 and 174.

A plurality of physiological sensor contacts are preferably replaceably mounted in base element 160 for engagement with a subject's hand when it is supported at support areas 162, 166, 168, 172 and 174.

In accordance with a preferred embodiment of the present invention, electro-dermal activity (EDA) sensor contacts 182, 186, 188, 192 & 194 are located at respective support areas 162, 166, 168, 172 and 174. In some specific non-limiting embodiments of the present invention, EDA sensor contacts 182, 186, 188, 192 & 194 may include a 030340 EDA Sensor Contacts, commercially available from Mindlife Solutions Ltd, Jerusalem Technological Park, Bldg. 1/C, Jerusalem, 96951 Israel.

In accordance with a preferred embodiment of the present invention, a photoplethysmograph (PPG) sensor contact 196 is located at support area 164. In some specific non-limiting embodiments of the present invention, photoplethysmograph (PPG) sensor contact 196 may preferably include a 6010-F, commercially available from HeartMath LLC, 14700 West Park Ave, Boulder Creek, Calif. 95006 USA,

One or more EDA processing circuit 200 receives outputs from sensor contacts 182, 186, 188, 192 & 194. In some specific non-limiting embodiments of the present invention, EDA processing circuit 200 may include a 030300 EDA, commercially available from Mindlife Solutions Ltd, Jerusalem Technological Park, Bldg. 1/C, Jerusalem, 96951 Israel.

A PPG processing circuit 202 receives outputs from sensor contact 196. In some specific non-limiting embodiments of the present invention, PPG processing circuit 202 include a 6010-M, commercially available from HeartMath LLC, 14700 West Park Ave, Boulder Creek, Calif. 95006 USA.

Preferably sweat wicking hand contact surface layer 210 is provided over base element 160 and is formed with apertures 212, 216, 218, 222, 224 & 226, for accommodating contacts 182, 186, 188, 192, 194 & 196.

As seen particularly in FIGS. 2D-4B, selectably inflatable finger immobilization elements 232, 234, 236 and 238 are mounted generally as shown facing respective support areas 162, 164, 166, 168 for immobilizing a subject's fingers when his hand in operative engagement with the hand engagement unit 150. FIGS. 4A and 4B show respective non-inflated and inflated states of one of the selectably inflatable elements 232-238.

Inflation and deflation controls 240 and 242 are preferably provided for enabling an operator to control inflation of selectably inflatable finger immobilization elements 232, 234, 236 and 238.

Further in accordance with a preferred embodiment of the invention, there is provided a selectable arm immobilizer 250 for selectably immobilizing an arm of said subject. Selectable arm immobilizer 250 preferably comprises a pair of upstanding plates 252 and 254, which are fixedly mounted onto common base 154. A pair of selectably inflatable arm immobilization elements 256 and 258 is mounted on inner facing surfaces of plates 252 and 254.

Inflation and deflation controls 260 and 262 are preferably provided for enabling an operator to control inflation of selectably inflatable arm immobilization elements 256 and 258.

Camera assembly 152 preferably comprises first and second thermal imaging cameras 270 and 272 which are preferably arranged to view the mouth and nose of the subject and the side neck region of the subject, respectively. Camera assembly 152 also comprises a motion detector 274. In some specific non-limiting embodiments of the present invention, a preferred motion detector 274 may include a cn 8537022606, commercially available from Prime Sense, 28 Habarzel St., Tel-Aviv 69710, Israel. The motion detector 274 preferably views the chest of the subject.

It is a particular feature of a preferred embodiment of the present invention that breathing rate is ascertained by use of a motion detector.

It is a further particular feature of a preferred embodiment of the present invention that at least one of heart rate, breathing rate and skin conductivity is ascertained by use of multiple different types of detectors. Examples of multiple different types of detectors which may be used for this purpose include:
a thermal camera and a motion detector, outputs of both of which are employed to ascertain breathing rate;
a PPG sensor and a thermal camera, outputs of both of which are employed to ascertain heart rate and which may be also used to calculate breathing rate; and one or more EDA sensors and a motion detector, outputs of both of which are employed to ascertain skin conductivity.

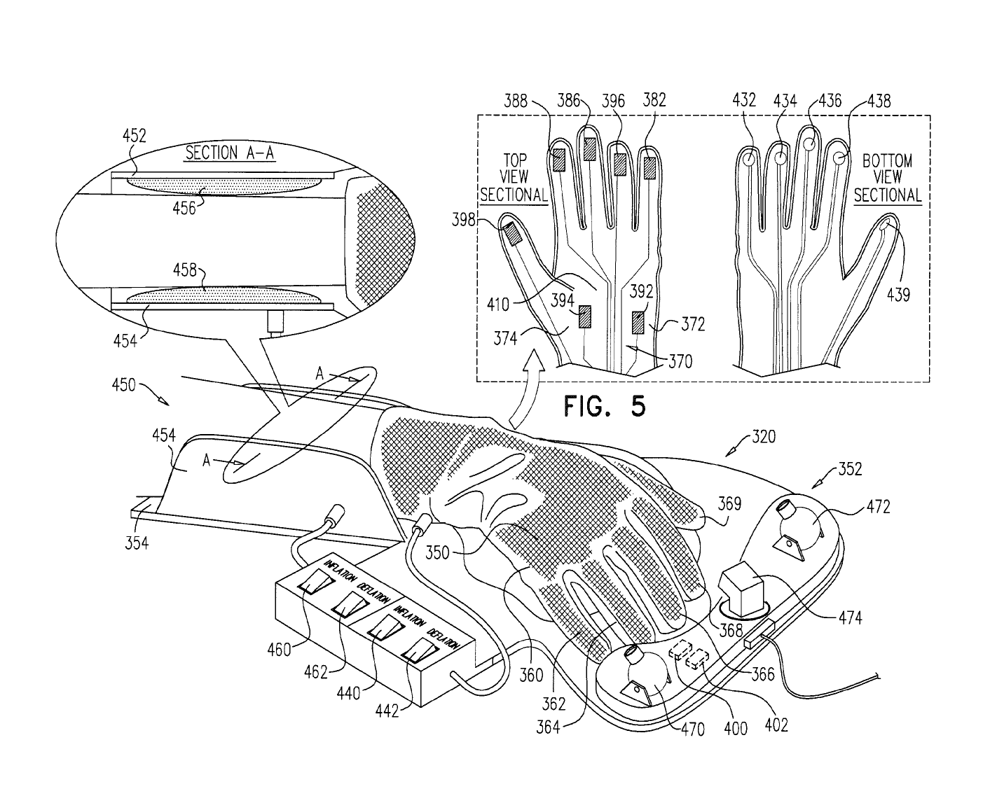

Reference is now made to FIG. 5, which is a simplified pictorial illustration of a subject observation subsystem 320, forming part of a computerized veracity testing system constructed and operative in accordance with another preferred embodiment of the present invention.

As seen in FIG. 5, the subject observation subsystem 320 includes a hand engagement unit 350 and a camera assembly 352, both of which are preferably mounted on a common base 354.

The hand engagement unit 350 preferably comprises a glove element 360, which is mounted onto common base 354. Glove element 360 preferably includes five finger engagement areas 362, 364, 366, 368 and 369 and a palm engagement area 370 including first and second palm support regions 372 and 374.

A plurality of physiological sensor contacts are preferably mounted in glove element 360 for engagement with a subject's hand when it is fully inserted into glove element 360.

In accordance with a preferred embodiment of the present invention, electro-dermal activity (EDA) sensor contacts 382, 386, 388, 392 & 394 are located at respective engagement areas 362, 366, 368, 372 and 374. In some specific non-limiting embodiments of the present invention, EDA sensor contacts 382, 386, 388, 392 & 394 may preferably include a 030340 EDA Sensor Contact, commercially available from Mindlife Solutions Ltd, Jerusalem Technological Park, Bldg. 1/C, Jerusalem, 96951 Israel.

In accordance with a preferred embodiment of the present invention, a photoplethysmograph (PPG) sensor contact 396 is located at engagement area 364. In some specific non-limiting embodiments of the present invention, PPG sensor contact 396 may preferably include a 6010-F, commercially available from HeartMath LLC, 14700 West Park Ave, Boulder Creek, Calif. 95006 USA.

Further in accordance with a preferred embodiment of the present invention, at least one additional sensor 398, such as a temperature sensor, is located at engagement area 369.

One or more EDA processing circuit 400 receives outputs from sensor contacts 382, 386, 388, 392 & 394. In some specific non-limiting embodiments of the present invention, EDA processing circuit 400 may preferably include a 030300 EDA, commercially available from Mindlife Solutions Ltd, Jerusalem Technological Park, Bldg. 1/C, Jerusalem, 96951 Israel.

A PPG processing circuit 402 receives outputs from sensor contact 396. In some specific non-limiting embodiments of the present invention, PPG processing circuit 402 may preferably include a 6010-M, commercially available from HeartMath LLC, 14700 West Park Ave, Boulder Creek, Calif. 95006 USA.

Preferably glove element 360 includes a sweat wicking hand contact surface layer 410.

Selectably inflatable finger immobilization elements 432, 434, 436, 438 and 439 are mounted generally as shown facing respective engagement areas 362, 364, 366, 368 and 369 for immobilizing a subject's fingers when his hand in operative engagement with the hand engagement unit 350.

Inflation and deflation controls 440 and 442 are preferably provided for enabling an operator to control inflation of selectably inflatable finger immobilization elements 432, 434, 436, 438 and 439.

Further in accordance with a preferred embodiment of the invention, there is provided a selectable arm immobilizer 450 for selectably immobilizing an arm of said subject. Selectable arm immobilizer 450 preferably comprises a pair of upstanding plates 452 and 454, which are fixedly mounted onto common base 354. A pair of selectably inflatable arm immobilization elements 456 and 458 is mounted on inner facing surfaces of plates 452 and 454.

Inflation and deflation controls 460 and 462 are preferably provided for enabling an operator to control inflation of selectably inflatable arm immobilization elements 456 and 458.

Camera assembly 352 preferably comprises first and second thermal imaging cameras 470 and 472 which are preferably arranged to view the mouth and nose of the subject and the side neck region of the subject, respectively. Camera assembly 352 also comprises a motion detector 474. The motion detector 474 preferably views the chest of the subject. Camera assembly 352 may include an ordinary video camera. In some embodiments, camera assembly 352 may include a depth camera such as, for example, the camera made by Prime Sense or the camera assembly in the Kinect computer game for the Xbox, or any other suitable depth camera. In some specific non-limiting embodiments of the present invention, camera assembly 352 may include an en 8537022606, commercially available from Prime Sense, 28 Habarzel St., Tel-Aviv 69710 Israel.

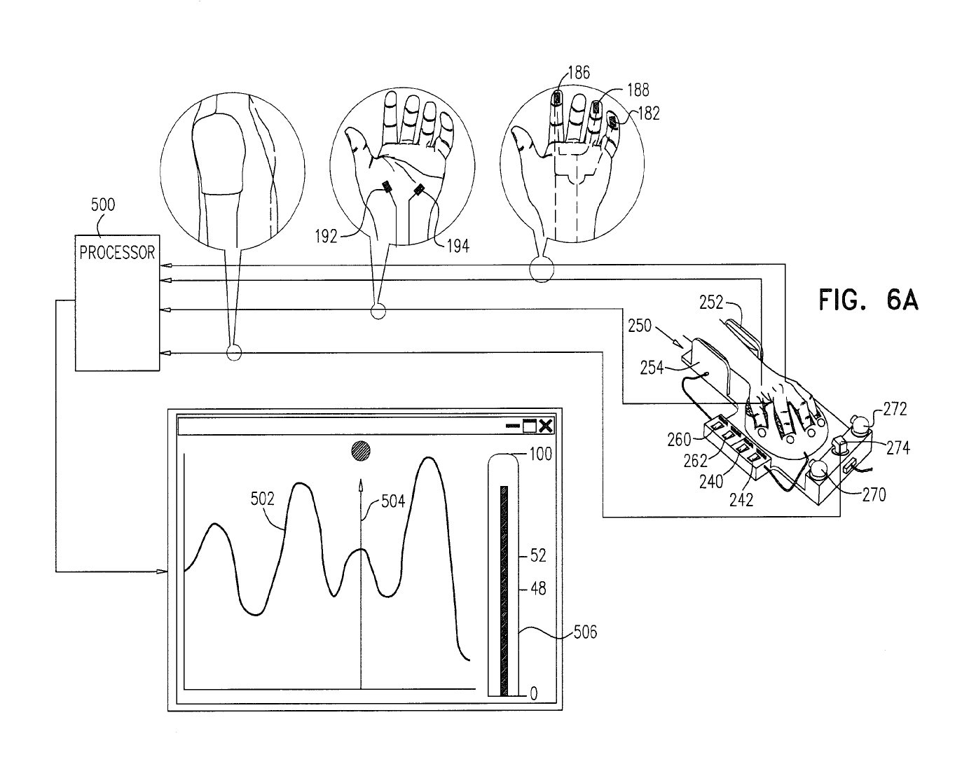

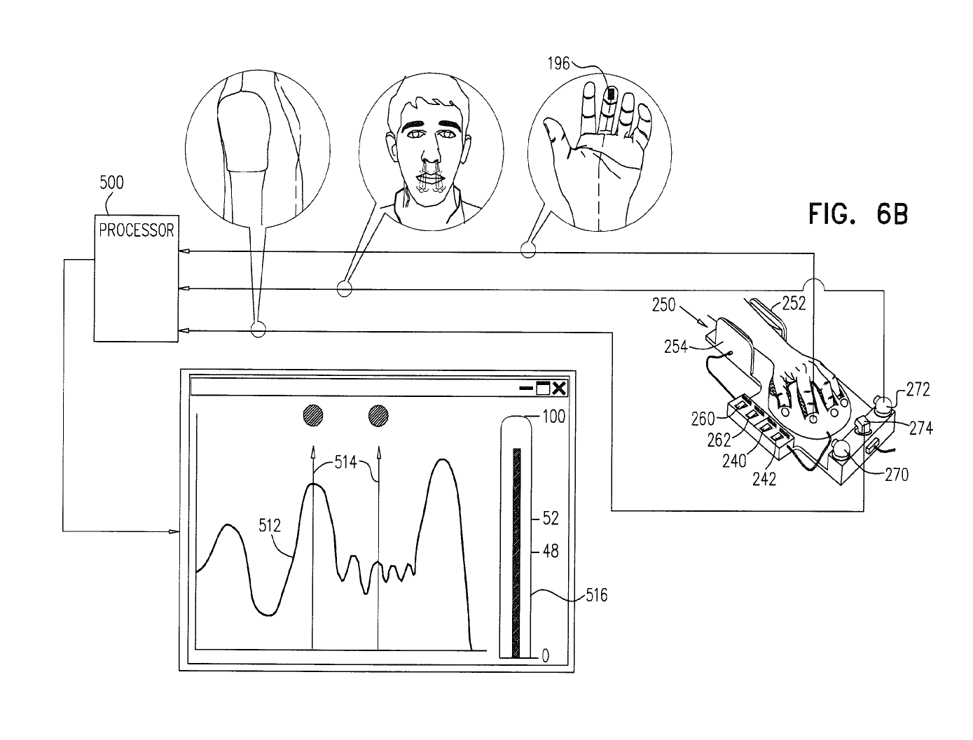

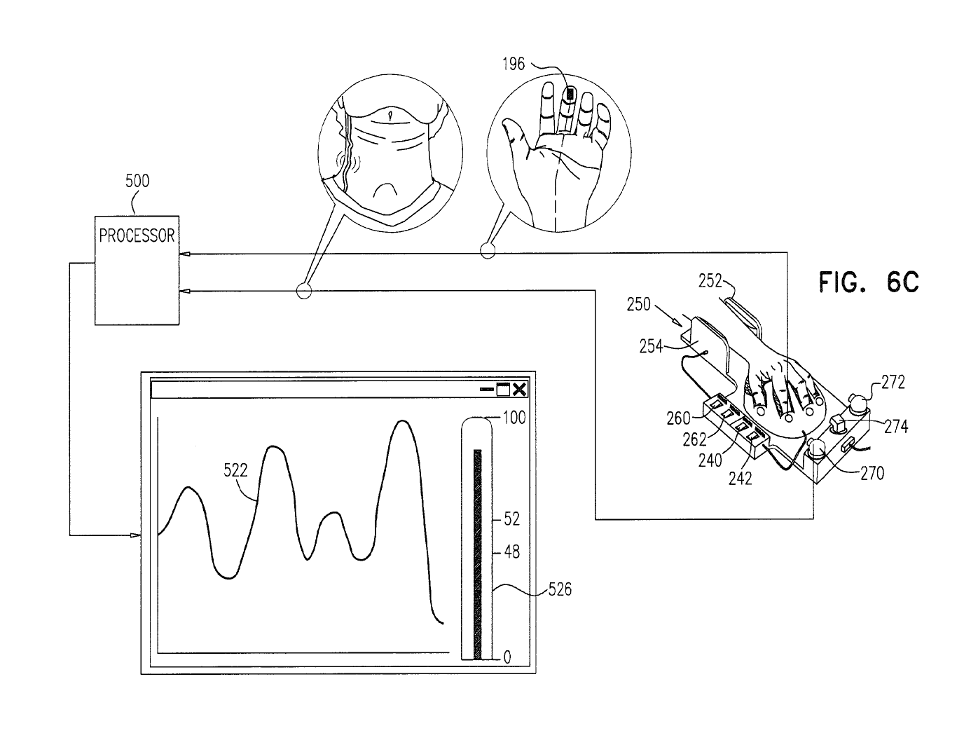

Reference is now made to FIGS. 6A, 6B and 6C, which are simplified illustrations of three different output functionalities employing multiple different types of sensors, preferably provided by the veracity testing system of a preferred embodiment of the present invention.

As noted above, it is a particular feature of the present invention that the subject observation subsystem of the present invention includes multiple different types of sensors providing outputs useful for ascertaining at least one of heart rate; breathing rate and skin conductivity and that there is provided an automatically operable computerized analysis subsystem responsive to said output of the subject observation subsystem for providing an indication relevant to veracity of the subject.

FIG. 6A illustrates a feature of the present invention wherein outputs from EDA finger sensors 182, 186 and 188 via multiple detection circuits, from EDA palm sensors 192 and 194 and from motion detector 274 are supplied to a processor 500, which may include processing circuits 200 and 202 (FIGS. 2A & 2B) or processing circuits 400 and 402 (FIG. 5). Processor 500 is operative to provide a skin conductivity change output 502, which typically appears on computer 122 (FIG. 1). In addition to the features described above, a particular feature of the conductivity change output 502 is the provision of one or more visual indications, here appearing as an arrow 504, which represents a sudden substantial chest movement of the subject, which is sensed by motion detector 274. In addition to the skin conductivity change output 502 there is preferably also provided a multi-parameter veracity indicator 506, which indicates the veracity of the subject based on the inputs provided to processor 500.

FIG. 6B illustrates a feature of the present invention wherein outputs from PPG sensor 196, from thermal camera 272 and from motion detector 274 are supplied to processor 500. Processor 500 is operative to provide a breathing rate change output 512, which typically appears on computer 122 (FIG. 1). In addition to the features described above, a particular feature of the breathing rate output 512 is the provision of one or more visual indications, here appearing as an arrow 514, which represents a sudden substantial chest movement of the subject, which is sensed by motion detector 274. In addition to the breathing rate change output 502 there is preferably also provided a multi-parameter veracity indicator 516, which indicates the veracity of the subject based on the inputs provided to processor 500.

FIG. 6C illustrates a feature of the present invention wherein outputs from PPG sensor 196 and from thermal camera 270 are supplied to processor 500. Processor 500 is operative to provide a heart rate change output 522, which typically appears on computer 122 (FIG. 1). In addition to the heart rate change output 522 there is preferably also provided a multi-parameter veracity indicator 526, which indicates the veracity of the subject based on the inputs provided to processor 500

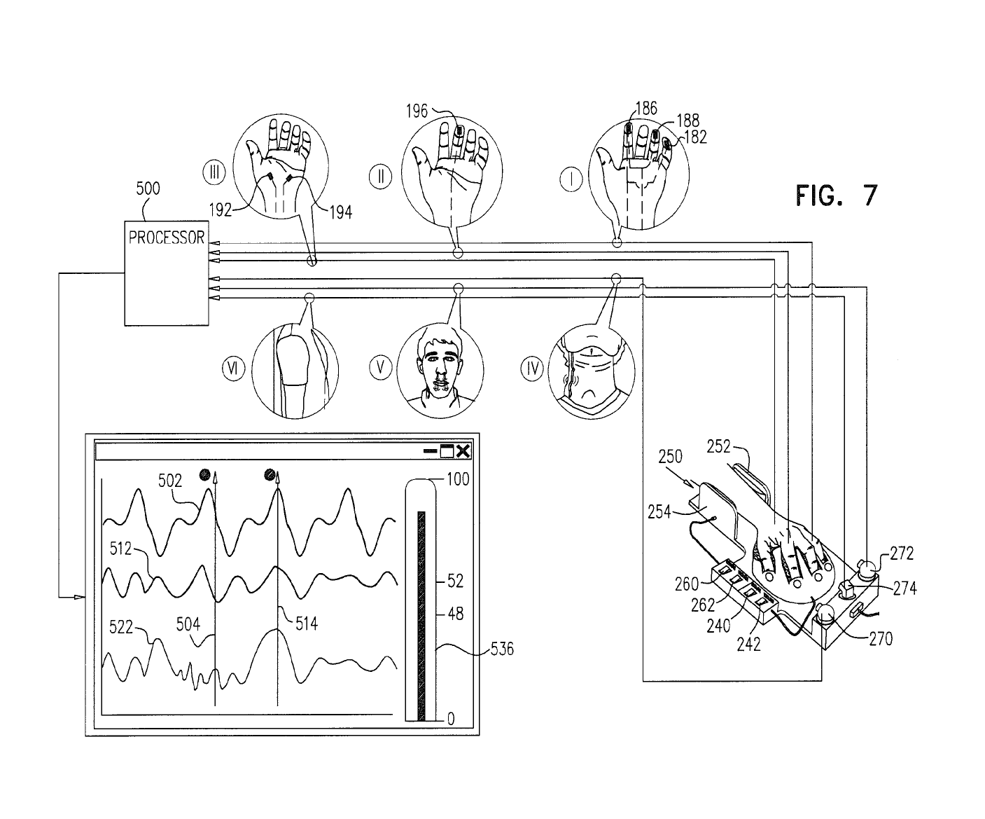

Reference is now made to FIG. 7, which is a simplified illustration of the operation of a veracity testing system providing the functionalities illustrated in FIGS. 6A, 6B and 6C. As seen in FIG. 7, in a preferred embodiment of the present invention, the following outputs are preferably provided to processor 500:

I. a first EDA detection circuit output from EDA finger sensors 182, 186 and 188;

II. a PPG detection circuit output from PPG finger sensor 196;

III. a second EDA detection circuit output from EDA palm sensors 192 and 194;

IV. an output from thermal camera 270 viewing the subject's neck;

V. an output from thermal camera 272 viewing the subject's face; and

VI. an output from motion detector 274 viewing the subject's chest.

Processor 500 is operative to provide a skin conductivity change output 502, a breathing rate change output 512 and a heart rate change output 522, which typically appear on computer 122 (FIG. 1). In addition to the various features described above, a particular feature of this embodiment of the invention is the provision of one or more visual indications, here appearing as arrows 504 and 514, which represent sudden substantial chest movements of the subject, which is sensed by motion detector 274. In addition to the outputs 502, 512 and 522, there is preferably also provided a multi-parameter veracity indicator 536, which indicates the veracity of the subject based on the various inputs provided to processor 500.

It is appreciated that processor 500 may also include additional processing circuits receiving outputs from the at least one additional sensor 398 (FIG. 5).

It is also appreciated that hand engagement units 150 and 350 may be configured for engagement with either the left hand or the right hand of the subject under observation.

According to some embodiments of the present invention, at least the perspiration level changes and/or heartbeat rate may be analyzed in order to detect a non-authentic response to a question. The perspiration level may be deduced according to changes in electric activity of the skin, such as the skin conductivity level. The skin conductivity and/or the heart beat rate may be monitored by various kinds of detectors, sensors and/or cameras as discussed in detail above. For example, the heart beat rate may be sensed by at least a photo-plethysmograph. For example, the perspiration level may be sensed by electric conductivity sensor. The monitored skin conductivity and/or heart rate changes may be processed by processor 500 as discussed above to produce output 502 and/or output 522, respectively.

The changes in perspiration level may be measured by the changes in electrical resistance of the skin, which may result from changes in sentimental perspiration (perspiration due to sentimental reasons). The meaning of the changes in heart rate may be concluded by identifying the frequency components of the heartbeat pulses by Fast Fourier Transform (FFT) and the ratio between the low frequency components and high frequency components, as well known in the art.

As discussed in detail herein, the method for recognizing a non-authentic response of a subject may include a questioning process, in which questions are presented to a subject, during which the perspiration level changes and/or heartbeat rate of the subject may be measured.

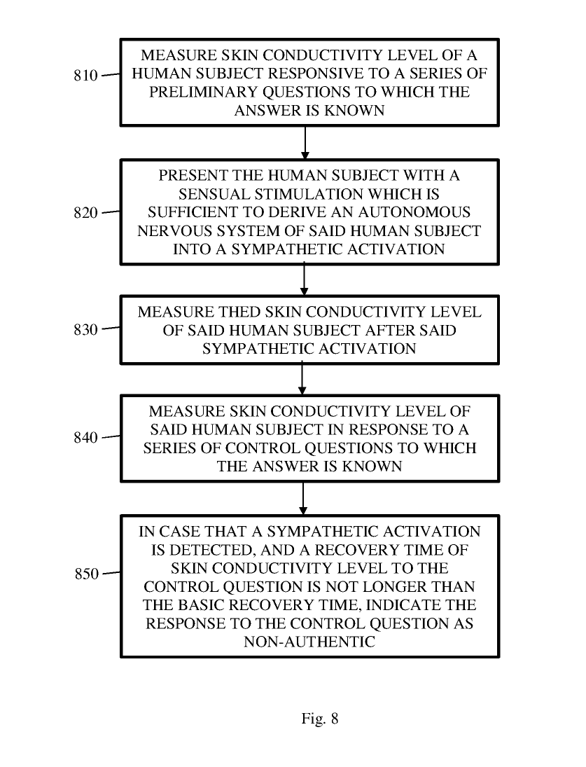

Reference is now made to FIG. 8, which is a schematic flowchart illustrating a method for recognizing a non-authentic response of a subject according to embodiments of the present invention. As indicated in block 810, the method may include, for example, measuring skin conductivity level of a human subject responsive to a series of preliminary questions to which the answer is known, wherein the measured skin conductivity level is defined as a baseline skin conductivity level. The questioning process may start with or may include, for example, at least two questions, for example in general subjects, with a known answer.

The relation between the basic/baseline conductivity of the subject and the inherent conductivity of the electric circuit has significant implication on the sensitivity of the result. Basically, the electric circuit is composed of two capacitors connected in parallel wherein one capacitor has constant capacity and the other one changes according to the perspiration level. In order to reach maximal sensitivity, we will compute the basic conductivity of the skin according to the at least two preliminary questions and the system according to embodiments of the present invention may adjust the level of the inherent conductivity of the circuit according to the computed basic conductivity of the skin of the specific subject. Accordingly, the method may include, for example, that the measuring of the skin conductivity level of said human subject is carried out using a skin conductivity measurement device having a variable resistance that is matched to the resistance of said human subject.

As indicated in block 820, the method may include presenting said human subject with a sensual stimulation which is sufficient to derive an autonomous nervous system of said human subject into a sympathetic activation. That is, the process may proceed with producing a stimulation which may include, for example, a stimulating question and/or sound and/or a strong sound. The purpose of this step is to identify the basic responsiveness of the subject both to non-stimulating questions and to questions that stimulate an uncontrollable physiological response. The preliminary questions and the following sensual stimulation may enable deduction of the automatic response level and the regular behavior of the subject under the presented questioning conditions.

After the stimulation, the basic/normal recovery time may be measured for the specific human subject. The basic recovery time may be defined as a time period in which the response level is reduced to a certain predetermined level after stimulation, for example, reduce to a level in the middle between the basic stress response and the baseline level. As indicated in block 830, the method may include, for example, measuring said skin conductivity level of said human subject after said sympathetic activation, wherein the measured skin conductivity level is defined as stress basic response level, and wherein a basic recovery time is defined as a time after which the value of the measurement reaches a specified value between the stress basic response level and the baseline skin conductivity level.

Then, a series of standardization/control questions may be presented to the subject, for example with varying answers length. As indicated in block 840, the method may include, for example, measuring skin conductivity level of said human subject in response to a series of control questions to which the answer is known. For example, informative questions may be asked, such as questions regarding name, phone numbers, workplace, address, etc. During the standardization/control questions stage, the measured values may be compared to the baseline response levels and recovery time after each response may be compared to the basic recovery time.

For example, in case a response to a control question has substantially the same level as the automatic response level and the recovery time is substantially the same or shorter than the basic recovery time, this may imply that the response non-authentic, for example that it is a response to an artificial stimulation and not a cognitive stimulation. In case that a sympathetic activation is detected, finding the authenticity of the response to the control question may be performed by comparing the recovery time of skin conductivity level for the control question with the basic recovery time. For example, an artificial stimulation may occur when the subject tries to imitate a response artificially or if he was affected by an irrelevant stimulation such as, for example, an arbitrary external sound. However, in case a there is a clear response to a control question, even with a lower level than the automatic response level, and the recovery time is significantly longer that the basic recovery time, this may imply a cognitive response and recovery difficulties, e.g. an authentic response.

Accordingly, as indicated in block 850, the method may include, for example, in case that a sympathetic activation is detected, finding the authenticity of the response to the control question by comparing the recovery time of skin conductivity level for the control question with the basic recovery time. For example, in case that a sympathetic activation is detected, and a recovery time of skin conductivity level to the control question is not longer than the basic recovery time, indicating the response to the control question as non-authentic.

In some embodiments of the present invention, the heartbeat of a subject may be measured during the process described herein with reference to FIG. 8, at least during the time period in which the series of control questions is asked, and the baseline structure of the subject's heartbeat coherence may be calculated. Measuring a heartbeat of a subject includes, in some embodiments of the present invention, measuring Heart Rate Variability (HRV). The calculated baseline coherence structure may include the general characteristic of frequencies found by fast Fourier transform (FFT) analysis of the HRV data, providing the separation between Very low frequencies (VLF), Low frequencies (LF) and High frequency (HF). Additionally, the fluctuation degree of the skin conductivity level may be measured during a same time period, and if during a response to a question the fluctuation degree is higher than a certain predetermined threshold of fluctuation and the heartbeat coherence is higher and/or substantially different in structure than the heartbeat coherence baseline, the responses to the questions during this time period may be suspected as non-authentic and/or may be indicated accordingly. For example, in some exemplary embodiments, if the fluctuation degree is higher than 10 and the percentage of time during which the subject was in high level of coherence was higher than 90, the subject's answers are suspected to be non-authentic. However, in most embodiments, the structure of the heartbeat coherence may also be taken into account. In some embodiments, the fluctuation degree may be measured, for example, by a biofeedback application, for example such as ProRelax developed by Mindlife.

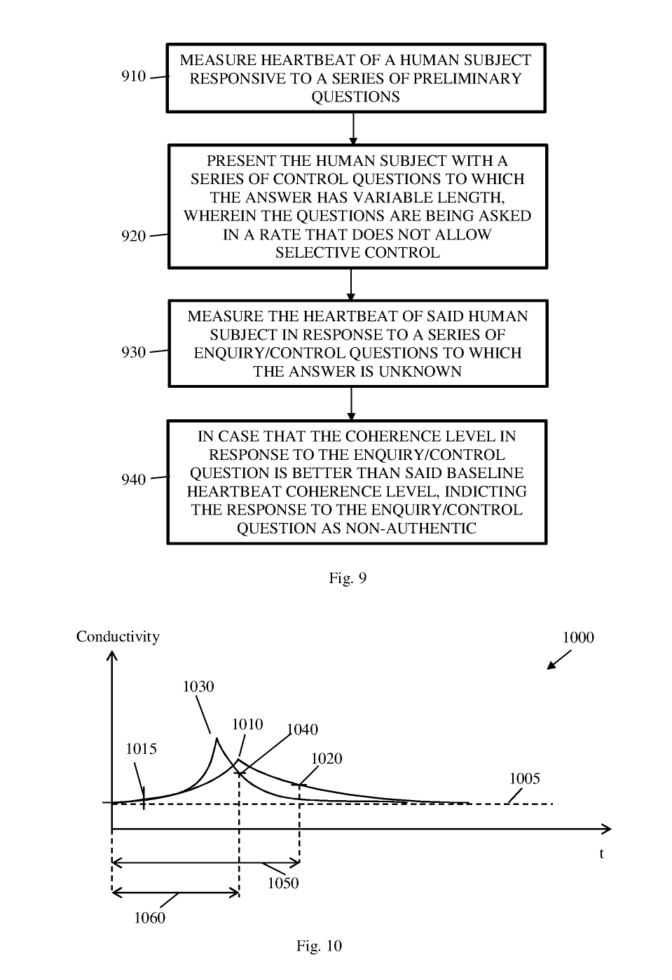

Reference is now made to FIG. 9, which is a schematic flowchart illustrating a method for recognizing a non-authentic response of a subject according to embodiments of the present invention. As indicated in block 910, the method may include, for example, measuring heartbeat of a human subject responsive to a series of preliminary questions to which the answer is known, to derive a level of coherence of the heartbeat being a baseline heartbeat coherence level. The preliminary questioning process may start with and/or may include, for example, at least two questions, for example in general subjects, with a known answer.

Then, a series of standardization/control questions may be presented to the subject, for example with varying answers length. As indicated in block 920, the method may include presenting said human subject with a series of control questions to which the answer has variable length, wherein the questions are being asked in a rate that does not allow said human subject to selectively control his or her respiratory system, so that the heartbeat coherence level of said human subject becomes unbalanced. During the preliminary questions stage, the basic coherency, i.e., the basic pattern of heart rate variability of the subject in peaceful periods of time, may be learnt. During the stage of control/standardization questions, the stress response coherency, i.e., the pattern of heart rate variability under conditions of irregular breathing, may be learnt.

As indicated in block 930, the method may include, for example, measuring said heartbeat of said human subject in response to a series of relevant enquiry questions, wherein said questions may be asked in a rate that allows said human subject to selectively control his or her respiratory system.

During the standardization/control questions stage, the measured values of heart rate changes coherency may be compared to the basic coherency. The authenticity of a response to a relevant question may be found by comparing the heartbeat coherence detected with the baseline heartbeat coherence and the unbalanced heartbeat coherence. For example, in some embodiments, in case the coherency during the questioning is significantly better than the basic coherency, this may imply that the subject tries to control the breathing rate in order to appear calm independently of his answers to the questions. The heart coherency and the perspiration measure may be sensed during the question and reply and immediately after the reply, for example in a time window of up to about twenty seconds. In case, during this period, the coherency is significantly worse than the basic coherency and the perspiration measure (skin conductivity) is higher than the basic response, this may imply that the replies given by the subject are highly non-authentic.

Accordingly, as indicated in block 940, the method may include, for example, finding the authenticity of a response to a relevant question by comparing the heartbeat coherence detected with the baseline heartbeat coherence and the unbalanced heartbeat coherence. In some embodiments, in case that the coherence level in response to the relevant enquiry question is better than said baseline heartbeat coherence level, the response to the relevant enquiry question may be indicated as non-authentic, e.g., the subject is trying to control his/her heartbeat in order to hide the authentic response to the relevant question.

In known methods, it is conventional to wait about twenty seconds or another constant predetermined period of time between two consecutive questions in order to allow full recovery between the questions. In contrast, embodiments of the present invention may enable continuous questioning wherein when a stressful response is not detected within a short period of time from the presentation of the last question such as, for example, four seconds, the questioning may proceed with the next question. This may enable much shorter questioning time and more subjects may be questioned in a certain period of time.

Reference is now made to FIG. 10, which is a schematic graph 1000 illustrating the method described with reference to FIG. 8, for recognizing a non-authentic response of a subject according to embodiments of the present invention. Graph 1000 represents skin conductivity as function of time. As described, a baseline skin conductivity level 1005 may be measured according to embodiments of the present invention. When stimulation is activated, such as, for example, an external physical excitement, at time 1015, a basic response level 1030 and a basic recovery time 1060 may be measured, wherein the basic recovery time is defined as a time after which the value of the measurement reaches a specified value 1040 between stress basic response level 1030 and the baseline skin conductivity level 1005.

Afterwards, a series of control questions may be presented and response levels 1010 may be measured. During the standardization/control questions stage, the measured values 1010 may be compared to the baseline response level 1030 and recovery time 1050 after each response 1010 may be compared to the basic recovery time 1060. Recovery time 1050 is defined as a time after which the value of the measurement reaches a specified value 1020 between response level 1010 and the baseline skin conductivity level 1005.

In the example of FIG. 10 it may be concluded, for example, that in case the recovery time of the skin conductivity level after the response to the control question is not longer than the basic recovery time 1060, the response to the control question may be suspected as non-authentic. However, in case a there is a clear response 1010 to a control question, even with a slightly lower level than automatic response level 1030, and recovery time 1050 is significantly longer that basic recovery time 1060, this may imply a cognitive response and recovery difficulties, which may imply, for example, an authentic response.

It will be appreciated by persons skilled in the art that the present invention is not limited by what has been particularly shown and described hereinabove. The scope of the present invention also includes combinations and subcombinations of various features described hereinabove as well as variations and modifications thereof which would occur to persons skilled in the art upon reading the foregoing description and are not in the prior art.

1. A method comprising:
measuring skin conductivity level of a human subject responsive to a preliminary questioning comprising a series of preliminary questions, wherein the measured skin conductivity level is defined as a baseline skin conductivity level;
presenting said human subject with a sensual stimulation which is sufficient to derive an autonomous nervous system of said human subject into a sympathetic activation;
measuring said skin conductivity level of said human subject after said sympathetic activation, wherein the measured skin conductivity level is defined as stress basic response level, and wherein a basic recovery time is defined as a time after which the value of the measurement reaches a specified value between the stress basic response level and the baseline skin conductivity level;
measuring skin conductivity level of said human subject in response to a series of control questions to which the answer is non-controversial and known to the subject; and
in case that a sympathetic activation is detected, finding the authenticity of the response to the control question by comparing the recovery time of skin conductivity level for the control question with the basic recovery time.

2. The method according to claim 1, wherein the measuring of the skin conductivity level of said human subject is carried out using a skin conductivity measurement device having a variable resistance that is matched to the resistance of said human subject.

3. The method according to claim 1, wherein the preliminary questioning includes at least two questions with a known non-controversial answer.

4. The method according to claim 1, further comprising after presenting the stimulation, deduction of the automatic response level and the regular behavior of the subject under the presented questioning conditions.

5. The method according to claim 1, further comprising, during the responding to a series of control questions, comparing the measured values to the baseline response levels and the recovery time after each response to the basic recovery time.

6. The method according to claim 1, further comprising measuring heartbeat coherence of the human subject during at least the series of control questions and calculating the subject's baseline structure of the subject heartbeat coherence.

7. A method according to claim 6, further comprising measuring the fluctuation degree of the skin conductivity, and if the fluctuation degree is higher than a certain predetermined threshold of fluctuation and the heartbeat coherence is higher and/or substantially different in structure than the heartbeat coherence baseline, indicating the responses to the control questions as suspected as non-authentic.

8. A method comprising:
measuring heartbeat of a human subject responsive to a series of preliminary questions to which the answer is non-controversial and known to the subject, to derive a level of coherence of the heartbeat being a baseline heartbeat coherence level;
presenting said human subject with a series of control questions to which the answer has variable length, wherein the control questions are being asked in a rate that does not allow said human subject to selectively control the subject's respiratory system, so that the heartbeat coherence level of said human subject becomes unbalanced;
measuring said heartbeat of said human subject in response to a series of questions relevant to the questioning subject; and
finding the authenticity of a response to a relevant question by comparing the heartbeat coherence detected with the baseline heartbeat coherence and the unbalanced heartbeat coherence.

9. The method according to claim 8, wherein the preliminary questioning includes at least two questions with a non-controversial known answer.

10. The method according to claim 8, wherein during the preliminary questions stage, the method further comprises learning the basic heart coherency of the specific subject, and during the stage of control questions, the method further comprises learning the stress response coherency.
